(19) United States
(12) Patent Application Publication
(10) Pub. No.: US 2026/0083814 A1
(43) Pub. Date: Mar. 26, 2026

(54) DIETARY SUPPLEMENT FORMULATION AND METHOD OF PREPARATION THEREOF

(71) Applicant: Darius Jones, Atlanta, GA (US)

(72) Inventor: Darius Jones, Atlanta, GA (US)

(21) Appl. No.: 18/896,966

(22) Filed: Sep. 26, 2024

(51) Int. Cl. A61K 36/9066; A61K 9/48; A61K 31/047; A61K 31/10; A61K 31/12; A61K 31/202; A61K 31/4525; A61K 31/593; A61K 31/714; A61K 33/06; A61K 36/185; A61K 36/324; A61K 36/67; A61P 29/00

(52) U.S. Cl. CPC A61K 36/9066; A61K 9/4833; A61K 31/047; A61K 31/10; A61K 31/12; A61K 31/202; A61K 31/4525; A61K 31/593; A61K 31/714; A61K 33/06; A61K 36/185; A61K 36/324; A61K 36/67; A61P 29/00

(57) ABSTRACT

The disclosure provides a dietary supplement formulation to alleviate sciatica pain, support nerve health, and enhance musculoskeletal health. The formula comprises a synergistic blend of natural ingredients, each selected for its specific properties. The formulation includes a Turmeric Extract (200 mg) for its anti-inflammatory and antioxidant effects, Myo-Inositol Powder (300 mg) for nerve signal transmission, MSM 80 Mesh (300 mg) for reducing inflammation and supporting connective tissue, and Black Currant Extract (250 mg) for its antioxidant properties. Omega 3 Fatty Acid (40 mg) provides anti-inflammatory and neuroprotective benefits, BioPerine (5 mg) enhances ingredient bioavailability, Magnesium (30 mg) supports nerve function and muscle relaxation, Vitamin D3 (10 mcg) is essential for bone health, Vitamin B12 (500 mcg) maintains healthy nerve cells, and Boswellia serrata Extract (100 mg) reduces inflammation.

### FIELD OF THE DISCLOSURE

The present disclosure relates to the field of dietary supplements, specifically to formulations designed to address sciatica pain, support nerve health, and enhance musculoskeletal health. More particularly, the disclosure relates to a dietary supplement comprising a synergistic blend of natural ingredients, including Turmeric Extract, Myo-Inositol Powder, MSM 80 Mesh, Black Currant Extract, Omega 3 Fatty Acid, BioPerine, Magnesium, Vitamin D3, Vitamin B12, and Boswellia serrata Extract. The formulation is designed to provide comprehensive relief from inflammation, improve blood circulation, and alleviate pain associated with sciatica, while also supporting overall nerve and musculoskeletal health. The supplement is encapsulated using an encapsulation machine, ensuring precise dosage and effective delivery of the active ingredients.

### BACKGROUND OF THE DISCLOSURE

Sciatica is a common condition characterized by pain radiating along the sciatic nerve, which extends from the lower back through the hips and buttocks and down each leg. This condition is often caused by inflammation or compression of the sciatic nerve due to various factors, including herniated discs, bone spurs, or muscle spasms. Traditional treatments for sciatica include pain medications, physical therapy, and in severe cases, surgery. However, these treatments often come with limitations, side effects, and varying degrees of effectiveness.

In recent years, there has been growing interest in the use of dietary supplements to manage sciatica pain and support overall nerve and musculoskeletal health. Natural ingredients, known for their anti-inflammatory, antioxidant, and neuroprotective properties, offer a promising alternative for individuals seeking to alleviate sciatica symptoms and improve their quality of life.

The prior art document ‘JP2009159873A’ discloses a nutritional supplement and health food that supplies at least one component. This component is added to existing food and ingested simultaneously with the food. The nutritional supplement and health food are produced using wheat flour and/or starch as the primary raw material. Prescribed nutritional components, food material, saccharide and/or dextrin, a leavening agent, and water are then added. The mixture is kneaded into dough and heat-dried using microwaves.

The present disclosure discloses a nutritional supplement which is added to the food and eaten simultaneously with the food. The supplement comprises Turmeric, MSM, Black Pepper, Magnesium, Vitamin B12, EPA, and DHA. Further, the supplement does not mention Boswellic Acid, Vitamin D3, Myo-Inositol Powder, and Black Currant in its composition.

Another prior art document ‘WO2023114885A1’ discloses a pet food composition comprising a fiber complex comprising NDF and crude fiber. Other embodiments of the present disclosure provide a pet food composition comprising: an anti-inflammatory component; from about 15 wt. % to about 25 wt. %, of NDF; and from about 10 wt. % to about 20 wt. % crude fiber; wherein the total fiber content of the pet food composition exceeds about 30 wt. %; and wherein the pet food composition improves HIP. In further embodiments, the NDF is greater than 20% wt. of the composition and/or the crude fiber is about 11% to about 20% wt. of the composition. In some embodiments, the pet food composition comprises about 21% NDF and about 13% crude fiber. Still further embodiments of the present disclosure comprise about 10% to about 50% protein, about 5% to about 50% fat, and/or about 6% to about 12% moisture.

The present disclosure discloses a composition for improving the health of animals. The supplements of the prior art are similar to the supplements of the present disclosure. The cited prior art comprises Turmeric, MSM, Magnesium, Vitamin D3, Vitamin B12, Inositol, EPA, and DHA. Further, the cited prior fails to disclose Boswellic Acid, Black Pepper, and black currant in its composition.

Another prior art document ‘US20110189161A1’ discloses a proprietary compositions and systems to modulate genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for the purposes of customizing a subject's nutritional supplement formulation to optimize specific health outcomes. Specific to this disclosure the utilization of certain known polymorphic genes associated with Substance Use Disorder (SUD) are analyzed to target certain genetic anomalies that lead to a high risk and predisposition to SUD. The genotypic patterns are then utilized to provide certain nutritional customized solutions especially related to the attenuation of aberrant abuse of physician prescribed narcotic pain medication across all pain conditions. A priority GENOPROFILE is measured and directs the customization of a subsequent nutraceutical to act as a therapeutic modality. Specifically, the treatment includes slow attenuation of the pain medication by incorporating orals (shakes, liquid beverages, pills, tablets, troche, ointments, etc.), Intramuscular, Intravenous, intra-rectal, and any form necessary to deliver a sufficient amount of an anti-craving and anti-stress nutraceutical. Moreover, the disclosure includes examples of novel analgesic ointments coupling Synaptamine and such analgesic and other anesthetic compounds including but not limited to Gabapentin, Ketamine, Baclofen, Ketoprofen, Amitriptyline, Lidocaine, Cyclobenzapine, Diclofenac, Menthol, Camphor and Capsaicin. The GENOPROFILE will be used to determine pain sensitivity Intolerance.

The present disclosure addresses the need for an effective, natural, and holistic approach to managing sciatica pain, supporting nerve health, and enhancing musculoskeletal health. The dietary supplement formulation combines carefully selected natural ingredients, each contributing to the overall efficacy of the product. The formulation includes Turmeric Extract, Myo-Inositol Powder, MSM 80 Mesh, Black Currant Extract, Omega 3 Fatty Acid, BioPerine, Magnesium, Vitamin D3, Vitamin B12, and Boswellia serrata Extract.

The selection of these ingredients is based on extensive research and their demonstrated benefits in reducing inflammation, supporting nerve function, and promoting musculoskeletal health. Turmeric Extract, for example, is well-known for its potent anti-inflammatory and antioxidant properties, which help alleviate inflammation and protect nerve cells from oxidative stress. Similarly, Myo-Inositol Powder supports nerve signal transmission and osmotic regulation, while MSM 80 Mesh provides anti-inflammatory and analgesic effects to support connective tissue integrity.

The formulation is manufactured in an FDA-certified facility that adheres to Good Manufacturing Practices (GMP), ensuring the highest standards of quality and safety. The manufacturing process includes sourcing high-quality, pure ingredients, accurately weighing and mixing these ingredients, encapsulating the mixture, conducting rigorous quality control checks, and packaging the final product.

The present disclosure represents a significant advancement in the field of dietary supplements for sciatica relief, nerve health, and musculoskeletal support. By providing a comprehensive, natural solution, the formulation offers a valuable alternative to traditional treatments, helping individuals manage their sciatica symptoms and improve their overall well-being.

To sum up, the existing processes have many limitations. Therefore, there is a need to provide an improved, simple, cost-effective, time-saving, energy-efficient, and generalized low-dose technology for the delivery of microbial bio-products to plants, which can be packaged in sachets, along with a method for their preparation.

### OBJECTIVES OF THE DISCLOSURE

The primary objective of the disclosure is to provide a dietary supplement formulation designed to effectively alleviate sciatica pain, support nerve health, and enhance musculoskeletal health.

Another objective of the present disclosure is to provide the dietary supplement formulation to ensure effective delivery and bioavailability of the active components of the formulation which is effectively absorbed and utilized by the body.17

Another objective of the present disclosure is to provide a consistent and high-quality supplement of the dietary supplement formulation.

Another objective of the present disclosure is to ensure the formulation achieves these goals through a synergistic blend of carefully selected natural ingredients, each contributing unique properties to enhance the overall effectiveness of the product.

### SUMMARY OF THE DISCLOSURE

The following summary is provided to facilitate an understanding of some innovative features unique to the disclosed embodiments and is not intended for the full description of the disclosure. A full appreciation of the various aspects of the preferred embodiments disclosed herein can be gained by taking the entire specification, claims, drawings, and abstract as a whole.

The present disclosure provides a dietary supplement formulated to alleviate sciatica pain, support nerve health, and enhance musculoskeletal well-being. The supplement features a blend of natural ingredients, including Turmeric Extract (200 mg) standardized to 95% curcuminoids for anti-inflammatory and antioxidant effects, Myo-Inositol Powder (300 mg) for nerve signal transmission, and MSM 80 Mesh (300 mg) for inflammation reduction and connective tissue support. Additional components include Black Currant Extract (250 mg) for its antioxidant properties, Omega 3 Fatty Acids (40 mg) for neuroprotection and inflammation reduction, and BioPerine Piper nigrum Fruit Extract (5 mg) to enhance ingredient bioavailability. Magnesium (30 mg), Vitamin D3 (10 mcg), Vitamin B12 (500 mcg), and Boswellia serrata Extract (100 mg) further support nerve function, muscle relaxation, and musculoskeletal health. The manufacturing process involves high-quality ingredient sourcing, precise formulation, encapsulation, and rigorous quality control to ensure a consistent and effective product, adhering to Good Manufacturing Practices (GMP).

Other features and advantages of the present disclosure will become apparent from the detailed description, taken in conjunction with accompanying drawings which illustrate, by way of example, the principles of the disclosure.

### BRIEF DESCRIPTION OF THE DRAWINGS

The drawings described herein are for illustrative purposes only of selected embodiments, not all possible implementations, and are not intended to limit the scope of the present disclosure. The disclosure itself, in terms of both its organization and method of operation, may be best understood by reference to the following detailed description, taken in conjunction with the accompanying drawings, in which:

FIG. 1 is a flow diagram representing the dietary supplement formulation for alleviating sciatica pain, supporting nerve health, and enhancing musculoskeletal health and each ingredient and its purpose are shown, with arrows indicating their respective functions, in accordance to the present disclosure.

FIG. 2 is a flow diagram representing the method for the preparation of a dietary supplement formulation, in accordance to the present disclosure.

In order to illustrate how the above-cited and other advantages and objects of the disclosure are obtained, a more detailed description of the disclosure, briefly described above, is provided with reference to the appended drawings. It will be recognized by a person of ordinary skill in the art, given the benefit of this disclosure, that the examples/results shown in the figures are not necessarily drawn to scale.

### DETAILED DESCRIPTION

The exemplary embodiments are provided to illustrate aspects of the disclosure, but the disclosure is not limited to any embodiment. The scope of the disclosure encompasses numerous alternatives, modifications, and equivalents; it is limited only by the claims.

Numerous specific details are set forth in the following description in order to provide a thorough understanding of the disclosure. However, the disclosure may be practiced according to the claims without some or all of these specific details. For the purpose of clarity, technical material that is known in the technical fields related to the disclosure has not been described in detail so that the disclosure is not unnecessarily obscured.

As used herein, the singular forms “a”, “an” and “the” designate both the singular and the plural, unless expressly stated to designate the singular only.

The present disclosure is illustrated with the help of the following examples, which are not intended to limit the scope of the disclosure and any such modification therein falls within the scope of this disclosure. It is to be understood that both the foregoing general description and the following detailed description are exemplary and explanatory only and are intended to provide further explanation of the subject matter.

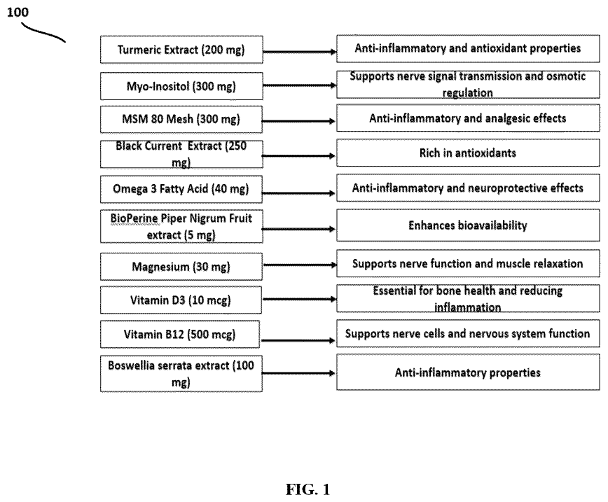

In one embodiment of the present disclosure, a dietary supplement formulation (A Formula for Sciatica Relief, Nerve Health, and Musculoskeletal Health) for alleviating sciatica pain, supporting nerve health, and enhancing musculoskeletal health is disclosed. This formulation comprises a Turmeric Extract in an amount of 200 mg, which provides anti-inflammatory and antioxidant properties to alleviate inflammation and protect nerve cells. It also includes Myo-Inositol Powder in an amount of 300 mg, supporting nerve signal transmission and osmotic regulation to reduce nerve pain and promote optimal nerve health. Furthermore, MSM 80 Mesh is included in an amount of 300 mg to deliver anti-inflammatory and analgesic effects, reducing inflammation and supporting connective tissue integrity. The formulation also contains Black Currant extract in an amount of 250 mg, rich in antioxidants to neutralize free radicals, reduce oxidative stress, and improve blood circulation. Omega 3 Fatty Acid in an amount of 40 mg, including eicosapentaenoic acid (EPA) and docosahexaenoic acid (DHA), provides anti-inflammatory and neuroprotective benefits. BioPerine Piper nigrum fruit extract in an amount of 5 mg enhances the bioavailability of other ingredients. Magnesium in an amount of 30 mg contributes to nerve function and muscle relaxation, while Vitamin D3 in an amount of 10 mcg is essential for bone health and reducing inflammation. Vitamin B12 in an amount of 500 mcg is vital for maintaining healthy nerve cells and supporting nervous system function. Lastly, Boswellia serrata Extract in an amount of 100 mg provides anti-inflammatory properties to address musculoskeletal health as shown in flowchart 100 of FIG. 1.

In another embodiment, the dietary supplement formulation (A Formula for Sciatica Relief, Nerve Health, and Musculoskeletal Health) as described above includes Turmeric Extract comprising 95% curcuminoids, which is included in an amount effective to reduce sciatic nerve inflammation. The Turmeric Extract is specifically extracted from the root of the Curcuma longa plant. Additionally, the Myo-Inositol Powder used is a form of Vitamin B8, and the MSM 80 Mesh is methylsulfonylmethane. The Black Currant extract is in a ratio of 4:1, is in powder form, and is extracted from the shrubs Ribes nigrum, specifically from the berry. The Omega 3 fatty acids, including EPA and DHA, are included in an amount effective to reduce inflammation and maintain nerve cell membrane integrity. The BioPerine Piper nigrum fruit extract is standardized to a minimum of 95% piperine and is in powder form. The Magnesium used in the formulation is in the form of Magnesium Citrate powder, and the Boswellia serrata Extract is standardized to 65% boswellic acid. Other ingredients in the formulation include Magnesium stearate and silicon dioxide.

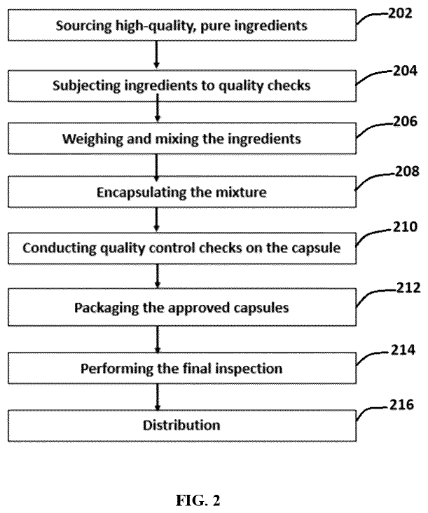

In yet another embodiment, a method for the preparation of the dietary supplement formulation (A Formula for Sciatica Relief, Nerve Health, and Musculoskeletal) Support is described. As shown in FIG. 2, the method comprises step 202 of sourcing high-quality, pure ingredients. The method further comprises step 204 of subjecting the sourced ingredients to quality checks to ensure they meet purity and quality standards through standardized testing procedures. The ingredients are then accurately weighed and uniformly mixed to achieve a consistent blend, at step 206. The uniformly mixed ingredient blend is filled into capsules using an encapsulation machine, at step 208. Quality control checks are conducted on the capsules, including assessments of weight uniformity, disintegration time, and other relevant quality parameters, at step 210. The approved capsules are then placed into suitable containers and labeled with the necessary product information, at step 212. A final inspection of the packaged product is performed to ensure it meets all quality and safety standards before distribution, at step 214. The weighing and mixing step involves using precision equipment to ensure accurate measurement and homogeneous distribution of the ingredients, at step 214. The encapsulation step uses an encapsulation machine designed to ensure the precise filling of the capsules with the mixed ingredient blend.

### Synergies Between Ingredients of the Formulation of the Present Disclosure:

The unique combination of ingredients in the dietary supplement formulation produces a synergistic effect that significantly enhances its overall efficacy in addressing sciatica pain, promoting nerve health, and supporting musculoskeletal well-being:
The blend of anti-inflammatory agents (Turmeric Extract, MSM, Omega 3 Fatty Acid, Magnesium, Vitamin D3, and Boswellia serrata Extract) offers a comprehensive approach to reducing inflammation, a critical factor in alleviating sciatica pain.
The inclusion of Myo-Inositol, Magnesium, and Vitamin B12 supports nerve signal transmission, muscle relaxation, and healthy nerve function, respectively, which helps alleviate sciatica symptoms.
BioPerine improves the bioavailability of the other ingredients, especially Turmeric Extract, ensuring maximum absorption and effectiveness of the formulation.
The antioxidant properties of Turmeric Extract, Black Currant Extract, and omega-3 Fatty Acid protect nerve and musculoskeletal tissues from oxidative stress and damage.
The synergistic interaction between Vitamin B12 and Myo-Inositol enhances nerve health by supporting myelin formation and nerve signal transmission, further reducing sciatica pain.

TABLE 1
The following ingredients used in the present disclosure:

| | | | Daily |
|---|---|---|---|
| Material | Dosage | | Value |
| Vitamin D3 (as Cholecalciferol) 100,000 iu/g | 10 | mcg | 50% |
| Vitamin B12 (as Methylcobalamin) | 500 | mcg | 20833% |
| Magnesium (as Magnesium Citrate Powder) | 30 | mg | 7% |
| Methylsulfonylmethane (MSM 80 Mesh) | 300 | mg | |
| Inositol | 300 | mg | |
| Black Currant Powdered Extract 4:1 | 250 | mg | |
| (Ribes nigrum) (Berry) | | | |
| Turmeric Extract (Curcuma longa) (Root) | 200 | mg | |
| (standardized to contain 95% curcuminoids) | | | |
| Boswellia Serrata Extract Standardized to 65% | 100 | mg | |
| Boswellic Acid | | | |
| Omega 3 Fatty Acid | 40 | mg | |
| Black Pepper Fruit Extract (BioPerine ®) | 5 | mg | |
| Yielding 95% Piperine | | | |

### Other Ingredients: Magnesium Stearate, Silicon Dioxide.

Table 2 shows the test results of the product/disclosure on people

| | | | | | | What was | |
|---|---|---|---|---|---|---|---|
| | | | | | | your | |
| | | | | What | | initial | |
| | | | | is your | | pain level | |
| | | | | daily | | before | Please |
| | | | | dosage | | using the | describe your |
| | | | How long | of the | How | formulation? | sciatica |
| | | | have you | formulation? | often | (1 = No | symptoms |
| | | Location | been | (e.g. 2 | do you | Pain, 5 | before using |
| First and | | (City, | using the | Capsules | take the | Worst Pain | the |
| Last Name | Age | State) | formulation | per day) | formulation? | Imaginable) | formulation. |
| Kathy | 61 | Covington, | 2 | 2 Caps | Daily | 5 | I would be in |
| Rouse | | Ga | Months | per day | | | lots of pain |
| | | | | | | | when I get up |
| | | | | | | | from sitting, |
| | | | | | | | standing for a |
| | | | | | | | period of time |
| | | | | | | | or lifting |
| | | | | | | | medium to |
| | | | | | | | heavy objects. |
| Dionte | 33 | Leesburg, | 1 | 2 | Daily | 3 | I would have a |
| Jones | | GA | Month | capsules | | | dull pain that |
| | | | | | | | would radiate |
| | | | | | | | on my left side |
| | | | | | | | down my leg. |
| Bauer | 60 | Douglasville, | 2 | 2 per | Daily | 5 | Sharp back |
| Harris | | GA | Months | day | | | pain in my |
| | | | | | | | lower back (L4 |
| | | | | | | | and L5); |
| | | | | | | | sporadic sharp |
| | | | | | | | pain down the |
| | | | | | | | leg after long |
| | | | | | | | standing or |
| | | | | | | | walking. |

| | | | How would | | |
|---|---|---|---|---|---|
| | | | you rate | How | |
| | | | your | satisfied | |
| | What is | | improvement | are you | |
| | your current | Please | or cure | with the | |
| | pain level | describe any | on a scale | formulation | |
| | after using the | improvements | of 1-10? | overall? | |
| | formulation? | in your | (1 = No | (1 = Very | Any |
| | (1 = No Pain, | symptoms after | Improvement, | Dissatisified, | additional |
| First and | 5 Worst Pain | using the | 5 = Completely | 5 = Very | comments or |
| Last Name | Imaginable) | formulation. | Cured) | Satissfied) | feedback? |
| Kathy | 1 | I'm able | 4 | 5 | I feel |
| Rouse | | to get up | | | that if I |
| | | from | | | continue |
| | | sitting | | | to take the |
| | | with no | | | formulation, |
| | | pain. | | | I will be |
| | | | | | completely |
| | | | | | healed. |
| Dionte | 1 | Yes, the | 4 | 5 | |
| Jones | | pain | | | |
| | | began to | | | |
| | | fade away | | | |
| | | and my | | | |
| | | mobility | | | |
| | | improved. | | | |
| | | I am now | | | |
| | | able to | | | |
| | | increase | | | |
| | | intensity of | | | |
| | | workout | | | |
| | | without | | | |
| | | the fear of | | | |
| | | pain | | | |
| Bauer | 2 | The pain | 4 | 5 | It would |
| Harris | | level has | | | be great |
| | | decreased | | | to have |
| | | significantly, | | | it in a |
| | | especially | | | powder |
| | | in the | | | form to |
| | | lower | | | mix with |
| | | back on a | | | water or |
| | | daily basis. | | | juice in |
| | | | | | the |
| | | | | | morning. |
| | | | | | I have |
| | | | | | some |
| | | | | | many |
| | | | | | other |
| | | | | | pills to |
| | | | | | take, so |
| | | | | | a powder |
| | | | | | mixture |
| | | | | | would |
| | | | | | be great. |

Although a preferred embodiment of the disclosure has been illustrated and described, it will at once be apparent to those skilled in the art that the disclosure includes advantages and features over and beyond the specific illustrated construction. Accordingly, it is to be noted that the scope of the disclosure is limited solely by the scope of the hereinafter appended claims, and not by the forgoing specification when interpreted in light of the relevant prior art.

1. A dietary supplement formulation for alleviating sciatica pain, supporting nerve health, and enhancing musculoskeletal health, comprising:
Turmeric Extract in an amount of 200 mg, providing anti-inflammatory and antioxidant properties to alleviate inflammation and protect nerve cells;
Myo-Inositol Powder in an amount of 300 mg, supporting nerve signal transmission and osmotic regulation to reduce nerve pain and promote optimal nerve health;
MSM 80 Mesh in an amount of 300 mg, delivering anti-inflammatory and analgesic effects to reduce inflammation and support connective tissue integrity;
Black Currant extract in an amount of 250 mg, rich in antioxidants to neutralize free radicals, reduce oxidative stress, and improve blood circulation;
Omega 3 Fatty Acid in an amount of 40 mg, including eicosapentaenoic acid (EPA) and docosahexaenoic acid (DHA), providing anti-inflammatory and neuroprotective benefits;
BioPerine Piper nigrum fruit extract in an amount of 5 mg, enhancing the bioavailability of other ingredients;
Magnesium in an amount of 30 mg, contributing to nerve function and muscle relaxation;
Vitamin D3 in an amount of 10 mcg, essential for bone health and reducing inflammation;
Vitamin B12 in an amount of 500 mcg, vital for maintaining healthy nerve cells and supporting nervous system function; and
Boswellia serrata Extract in an amount of 100 mg, providing anti-inflammatory properties to address musculoskeletal health.

2. The dietary supplement formulation of claim 1, wherein the turmeric extract comprises 95% curcuminoids and is included in an amount effective to reduce sciatic nerve inflammation.

3. The dietary supplement formulation of claim 1, wherein the turmeric extract is extracted from the root of Curcuma longa plant.

4. The dietary supplement formulation of claim 1, wherein the myoinositol powder is a Vitamin B8.

5. The dietary supplement formulation of claim 1, wherein the MSM 80 mesh is methylsulfonylmethane.

6. The dietary supplement formulation of claim 1, wherein the black currant extract is in a ratio of 4:1.

7. The dietary supplement formulation of claim 1, wherein the black currant extract is in the powder form.

8. The dietary supplement formulation of claim 1, wherein the black currant extract is extracted from the shrubs Ribes nigrum i.e. berry.

9. The dietary supplement formulation of claim 1, wherein the omega 3 fatty acids, including EPA and DHA, are included in an amount effective to reduce inflammation and maintain nerve cell membrane integrity.

10. The dietary supplement formulation of claim 1, wherein the BioPerine Piper nigrum fruit extract is standardized to a minimum of 95% piperine.

11. The dietary supplement formulation of claim 1, wherein BioPerine Piper nigrum fruit extract is in a powder form.

12. The dietary supplement formulation of claim 1, wherein the magnesium is a Magnesium Citrate powder.

13. The dietary supplement formulation of claim 1, wherein the Boswellia serrata Extract is standardized to 65% boswellic acid.

14. The dietary supplement formulation of claim 1, wherein the other ingredients are Magnesium stearate and silicon dioxide.

15. A method for the preparation of a dietary supplement formulation, comprising:
sourcing high-quality, pure ingredients that are subjected to quality checks to ensure they meet purity and quality standards through standardized testing procedure;
weighing and mixing the ingredients, wherein the ingredients are accurately weighed and uniformly mixed to achieve a consistent blend;
encapsulating the mixture, wherein the uniformly mixed ingredient blend is filled into capsules using an encapsulation machine;
conducting quality control checks on the capsules, wherein the checks include assessing weight uniformity, disintegration time, and other relevant quality parameters;
packaging the approved capsules, wherein the capsules are placed into suitable containers and labeled with the necessary product information; and
performing a final inspection of the packaged product to ensure it meets all quality and safety standards before distribution.

16. The method of claim 15, wherein the weighing and mixing step involves using precision equipment to ensure accurate measurement and homogeneous distribution of the ingredients.

17. The method of claim 15, wherein the encapsulation step uses an encapsulation machine designed to ensure precise filling of the capsules with the mixed ingredient blend.
